(19) United States
(12) Patent Application Publication
(10) Pub. No.: US 2005/0277696 A1
(43) Pub. Date: Dec. 15, 2005

(54) Novel use of cytotoxic drugs for treatment and prophylasxis of aging diseases by reversing the loss of elasticity of epithelial cells due to aging

(76) Inventors: Igor Sokolov, Potsdam, NY (US); Craig Woodworth, Potsdam, NY (US)

Correspondence Address: HISCOCK & BARCLAY, LLP, 2000 HSBC PLAZA, ROCHESTER, NY, 14604-2404, US

(21) Appl. No.: 11/150,973

(22) Filed: Jun. 13, 2005

Related U.S. Application Data
(60) Provisional application No. 60/579,165, filed on Jun. 14, 2004.

(51) Int. Cl. A61K 31/19

(52) U.S. Cl. 514/575

(57) ABSTRACT

A method for treatment of age-related symptoms of epithelial tissues that includes the administration of an agent that disrupts F-actin polymerization in an amount effective to reduce rigidity in epithelial cells.

### CROSS-REFERENCE TO RELATED APPLICATIONS

This application claims priority from U.S. Provisional Patent Application Ser. No. 60/579,165, filed Jun. 14, 2004.

### FIELD OF THE INVENTION

This invention relates to the use of inhibitors to prevent the increased polymerization of microfilaments in older epithelial cells thereby increasing their elasticity and preventing the side effects and diseases related to cell aging and rigidity.

### BACKGROUND OF THE INVENTION

The increase of rigidity of human epithelial tissues with aging has been implicated in the progression of many diseases associated with aging such as vascular diseases, kidney disease, cataracts, Alzheimer's Dementia, complications of diabetes and cardiomyopathies. It was commonly believed that the increase in rigidity resulted exclusively from cross-polymerization of collagen and elastin in the extra-cellular matrix. Many treatments for age-related diseases are based on preventing such cross polymerization. However, research has recently shown that, in addition to cross polymerization in the extra-cellular matrix, epithelial cells become considerably more rigid after aging. Apart from contributing to the increase of rigidity of epithelial tissue as a whole, more rigid cells can also inhibit important epithelial functions such as endocytosis, movement, and gene expression.

Previous research coupled with the use of atomic force microscopy (AFM) has disclosed a positive correlation between increased cell rigidity in aged cells and the surface density of cytoskeletal fibers. The use of AFM in combination with standard immunofluorescence microscopy has revealed that the majority of these fibers are microfilaments, i.e., F-actin, a protein molecule. Therefore, there is an identified need to provide a method for the inhibition of polymerization of F-actin molecules at the cellular level.

### SUMMARY OF THE INVENTION

The present invention provides a therapeutic method comprising the use of a therapeutic agent that will slow the cytoskeletal polymerization rate, specifically the polymerization of F-actin molecules, within the epithelial cells as a measure to control aging related diseases.

The preferred method of administration for inhibiting polymerization is the use of a topical cream that would be applied directly to the skin over the entire body or in areas most susceptible to accelerated polymerization of the epithelial cells or are simply more prone to malignant skin diseases. In addition, administration of cytotoxins or other agents in low amounts may serve as a preventative or therapeutic measure against many tissue related ailments including, but not limited to, epithelial carcinoma or other forms of malignant or pre-malignant diseases, tissue diseases that are either malignant or pre-malignant, infections to epithelial tissue such as infections to the skin, anogential tract and respiratory tract, impairment of glandular tissues due to inadequate production of mucous or secretions in the digestive system, wound related issues due to problems such as extensive fibrosis, ulcer formation or cataracts.

### DETAILED DESCRIPTION

The treatment of aged epithelial cells with cytotoxic drugs such as Cytochalasin B or other such drugs that inhibit polymerization of F-actin, will restore low rigidity to levels observed in cultures of young cells. The use of such drugs has been long known in areas such as cancer treatment, however, the drugs in cancer treatments are used in much higher concentrations so as to selectively destroy cancer cells that divide more rapidly than normal cells. In the present invention, the drugs are administered in much lower concentrations so as to inhibit F-actin polymerization while not destroying a significant number of cells.

However, the issue is complicated because each epithelial cell has three distinctive regions of rigidity: the area above the nucleus, the cytoplasm, and the cell edge. All three areas have quite distinctive cytoskeletal content. Fortunately, when using an appropriate treatment, one may simultaneously enhance elasticity of older cells to that of a younger cell in all three areas of rigidity.

Preferred inhibitors include, for example, Cytochalasin D, B and Latrunculin A, B, Jasplakinolide or other such agents which will inhibit the polymerization of F-actin. The administration of such a therapeutic agent is effective to slow the level of polymerization of F-actin and thus allow older cells, which contain a higher number of F-actin fibers and are therefore more rigid, to become increasingly elastic and more like younger cells, which contain fewer F-actin fibers.

Determining the optimal concentration of a cytoskeletal inhibitor so it will effectively inhibit the polymerization of F-actin while not destroying the cells is necessary for each individual drug as no two inhibitors are alike. Therefore, before the comparison of results based on rigidity of older cells against younger and more elastic cells, the proper concentration of the drug must be found. Dissolved in an appropriate solvent (for example, dimethylsulfoxide for Cytochalasin D, B and Latrunculin A, B, Jasplakinolide), the drug should be added to the cell culture medium. By testing various concentrations of the drug, one can find the maximum concentration that does not decrease cell number noticeably over several population doublings in the cell culture relative to cultures without the drug. The amount of the drug should not be in such a high concentration that would result in the destruction of the cells. As an example, when the treatment of the old cells is done with Cytochalasin B, a cell permeable fungal toxin which binds to the barbed end of actin filaments and results in the inhibition of both the association and dissociation of its subunits, a 4 μg/mL solution of Cytochalasin B in the medium was optimal to treat viable cells for 12 hours. This concentration was determined by performing experiments to measure clonal growth of human epithelial cells in cell culture over 7 days in different concentrations of the drug. The number of surviving colonies of cells was counted to determine the range of concentrations that were non-toxic or non-cytostatic. This range was from 1.0 to 10 μg/mL. The determination of the appropriate concentration of a cytotoxic drug is dependent upon the particular drug of choice, but one skilled in the art would recognize methods to determine the appropriate drug concentration.

An atomic force microscope (AFM) can be used to analyze the rigidity of all cells in HBSS solution in-vitro. Using standard known procedures, the AFM should be calibrated to do quantitative force measurements needed for obtaining information on cell rigidity. During measurement for rigidity, a 5 μm diameter silica ball is glued to the cantilever by epoxy resin. This is the essential part that allows one to reliably measure the cell rigidity.

For AFM analysis, all treated cells must be first prepared by washing twice by Hank's Balanced Salt Solution (HBSS), then treated overnight at 4° C. with a solution of 0.5% Triton X-100 detergent mixed with buffer (0.14 M NaCl, 5 nM MgCl₂) in order to solubilize the plasma membrane and organelles and remove cytoplasmic proteins. The remaining cytoskeletal network is washed twice for 2 min in the buffer and then fixed in the buffer with 1% formalin for 10 min. The fixative should then be removed and the cells should be again washed twice with HBSS. To prepare for analysis, the plates should be stained with a 10 μL methanolic stock solution of Alexa Fluor 488 phalloidin in 200 μL in HBSS. The cells are again left overnight at 4° C. with the staining stock solution. The fixative is removed and the cells are again washed with HBSS and then ultrapure water. Once washing is complete, the cells are dried under ambient conditions for analysis.

Determining the rigidity of the cells is done by measuring the Young's modulus over different points on the individual cell. The Young's modulus is a numerical representation of elasticity measured by the stress over the strain. Force curves must be collected over areas sufficient to represent the entire cell. From these force curves, the optimal concentration and the time of treatment may be found for a specific inhibitor. The optimal concentration should be below the maximum concentration as defined above.

In the clinical setting, after determining the optimal concentration for a specific inhibitor, one skilled in the art would recognize that various methods could be employed to apply a specific inhibitor either topically, by ingestion, by injection or other forms of application that will slow the cytoskeletal polymerization rate within the epithelial cells as a prophylactic measure to control aging related diseases in those members who are highly susceptible to such diseases. For instance, a predetermined amount of cycloskeletal inhibitor can be applied in a liquid or cream form to biologically slow the F-actin polymerization process. The inhibitor is of a concentration sufficient to inhibit the additional polymerization of the F-actin, but not sufficient to damage or destroy the cell. In the case of Cytochalasin B, a concentration of 4 μg/mL is sufficient.

One skilled in the art would also recognize that a cytoskeletal inhibitor can be applied via injection directly to the epithelial layer of an internal organ to inhibit cytoskeletal polymerization of epithelial cells within internal organs. Such a method would be a responsive measure to repair ageing related diseases.

While the invention has been described with reference to particular embodiments, it will be understood by those skilled in the art that various changes may be made and equivalents may be substituted for elements thereof without departing from the scope of the invention. In addition, many modifications may be made to adapt a particular situation or material to the teachings of the invention without departing from the scope of the invention.

Therefore, it is intended that the invention not be limited to the particular embodiments disclosed as the best mode contemplated for carrying out this invention, but that the invention will include all embodiments falling within the scope and spirit of the appended claims.

1) A method for treatment of age related symptoms of epithelial tissues comprising:
a) administering an agent that disrupts F-actin polymerization in an amount effective to reduce rigidity in epithelial cells.

2) The method as recited in claim 1, wherein said agent is selected from the group comprising Cytochalasin D, B and Latrunculin A, B, Jasplakinolide, and similar drugs inhibiting polymerization of F-actin in cells.

3) The method as recited in claim 1 where the agent is Cytochalasin B in a concentration of 1.0-10.0 μg/mL

4) A method of determining appropriate concentrations of therapeutic compounds that will lead to the impairment of polymerization of F-actin, which comprises
a) preparation of both young and old cells where young cells are maintained for less than 3 passages in-vitro and old cells are maintained for 8-12 passages in-vitro;
b) performing an analysis of cell rigidity;
c) a titration of cytotoxic drug concentration to determine the optimal concentration for the impairment of polymerization of F-actin.

5) A method as recited in claim 4, wherein the rigidity is determined through the use of an atomic force microscope.
